(19) United States
(12) Patent Application Publication
(10) Pub. No.: US 2011/0039877 A1
(43) Pub. Date: Feb. 17, 2011

(54) USE OF 3, 11B-CIS-DIHYDROTETRABENAZINE IN THE TREATMENT OF MULTIPLE SCLEROSIS AND AUTOIMMUNE MYELITIS

(75) Inventor: Andrew John Duffield, Hertfordshire (GB)

Correspondence Address: SCHWEGMAN, LUNDBERG & WOESSNER, P.A., P.O. BOX 2938, MINNEAPOLIS, MN, 55402, US

(73) Assignee: Biovail Laboratories International (Barbados) S.R.L., Christ Church (BB)

(21) Appl. No.: 12/740,786

(22) PCT Filed: Oct. 29, 2008
(86) PCT No.: PCT/GB2008/051017

§ 371 Date: Oct. 25, 2010

(30) Foreign Application Priority Data
Nov. 2, 2007 (GB) 0721669.0

(51) Int. Cl. A61K 31/4375; C07D 455/06; A61P 25/00; A61P 29/00; A61P 1/08; A61P 21/00; A61P 25/28; A61P 25/24; A61P 13/00; A61P 43/00

(52) U.S. Cl. 514/294; 546/95

(57) ABSTRACT

The invention provides a compound for use in treating multiple sclerosis wherein the compound is a 3, 11b-cis-dihydrotetrabenazine of the formula (Ia): or a pharmaceutically acceptable salt thereof.

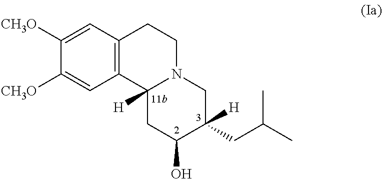

This invention relates to the use of a dihydrotetrabenazine in treating multiple sclerosis.

### BACKGROUND OF THE INVENTION

Multiple Sclerosis (MS) is a disabling neurological condition characterised by gradual destruction of the myelin sheath, a protective fatty layer that surrounds nerve fibres of the central nervous system. As a consequence of the damage to the myelin sheath, the nerve fibres can no longer effectively conduct electrical signals and this gives rise to a variety of symptoms, including changes in sensation, visual problems, muscle weakness, depression, difficulties with coordination and speech, severe fatigue, cognitive impairment, problems with balance, overheating, pain, and urinary and faecal incontinence. In more severe cases, MS will cause impaired mobility and disability.

MS is generally categorized as an autoimmune disease which results from attacks by an individual's immune system on the nervous system.

Multiple sclerosis can be categorised into three types, relapsing-remitting MS, secondary progressive MS and primary progressive MS. In around 80% of MS sufferers, the MS starts off as a relapsing and remitting condition which means that there are periods of relapse, when symptoms flare up, often quite suddenly, and then periods of remission, when symptoms improve. The periods between relapses can be highly unpredictable and often several years may pass between relapses.

After an initial period of relapsing-remitting MS, patients may progress to secondary progressive MS which involves the gradual accumulation of neurological disability, without remission, despite a reduction in the frequency of relapse. About half of people who have relapsing-remitting MS go on to the secondary progressive stage in the first 10 years.

The third type of MS, primary progressive MS, afflicts about 10% of MS patients. With this type of MS, there are no periods of remission and the disease gets gradually worse from the start. This causes increasing disability, and can reduce life expectancy.

There is currently no cure for multiple sclerosis but a number of different types of drugs are used to control or manage the symptoms, of which the most popular are anti-inflammatory steroids such as methyl prednisolone. Steroids are typically used to treat relapses but are not believed to alter the course of the disease. Largely because of the side effects, it is generally recommended not to use steroids for more than about three weeks at a time and for no more than about three courses per year. Side effects caused by steroids include stomach irritation, such as indigestion and heartburn, stomach ulcers, mood changes or mood swings, insomnia, nausea, bone-thinning osteoporosis, cataracts, weight gain, swelling and obesity, acne and diabetes. Steroids are generally suitable for treating only about 10-20% of relapses.

Non-steroidal anti-inflammatory drugs (NSAIDs) have been used to alleviate or manage some of the symptoms of MS but, again, they have no effect on the course of the disease. Moreover, they have well known side effects such as gastric irritation and can cause gastric bleeding and stomach ulcers.

A number of treatments, including β-Interferon and Copaxone, have been licensed as “disease modifying” therapies reducing the frequency of MS relapses and providing a modest benefit in terms of disability progression. However, at present, there remains a need for alternative, more effective symptomatic and disease modifying treatments for MS that lack the side effects associated with existing drug treatments.

Tetrabenazine (Chemical name: 1,3,4,6,7,11b-hexahydro-9,10-dimethoxy-3-(2-methylpropyl)-2H-benzo(a)quinolizin-2-one) has been in use as a pharmaceutical drug since the late 1950s. Initially developed as an anti-psychotic, tetrabenazine is currently used in the symptomatic treatment of hyperkinetic movement disorders such as Huntington's disease, hemiballismus, senile chorea, tic, tardive dyskinesia and Tourette's syndrome, see for example Jankovic et al., Am. J. Psychiatry. (1999) August; 156(8):1279-81 and Jankovic et al., Neurology (1997) February; 48(2):358-62.

The primary pharmacological action of tetrabenazine is to reduce the supply of monoamines (e.g. dopamine, serotonin, and norepinephrine) in the central nervous system by inhibiting the human vesicular monoamine transporter iso form 2 (hVMAT2). The drug also blocks postsynaptic dopamine receptors.

Tetrabenazine is an effective and safe drug for the treatment of a variety of hyperkinetic movement disorders and, in contrast to typical neuroleptics, has not been demonstrated to cause tardive dyskinesia. Nevertheless, tetrabenazine does exhibit a number of dose-related side effects including causing depression, parkinsonism, drowsiness, nervousness or anxiety, insomnia and, in rare cases, neuroleptic malignant syndrome.

The central effects of tetrabenazine closely resemble those of reserpine, but it differs from reserpine in that it lacks activity at the VMAT1 transporter. The lack of activity at the VMAT1 transporter means that tetrabenazine has less peripheral activity than reserpine and consequently does not produce VMAT1-related side effects such as hypotension.

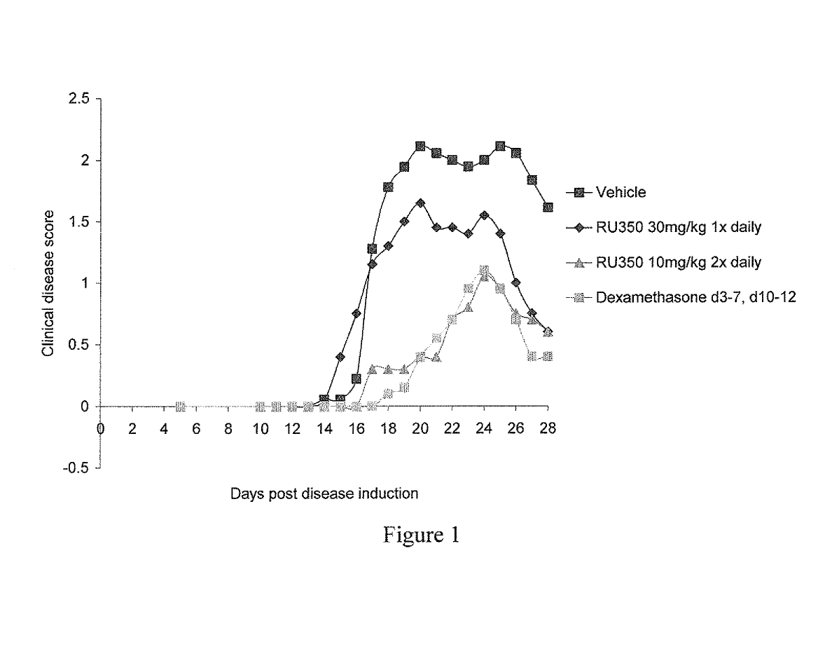

The chemical structure of tetrabenazine is as shown in FIG. 1 below.

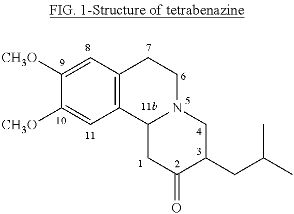

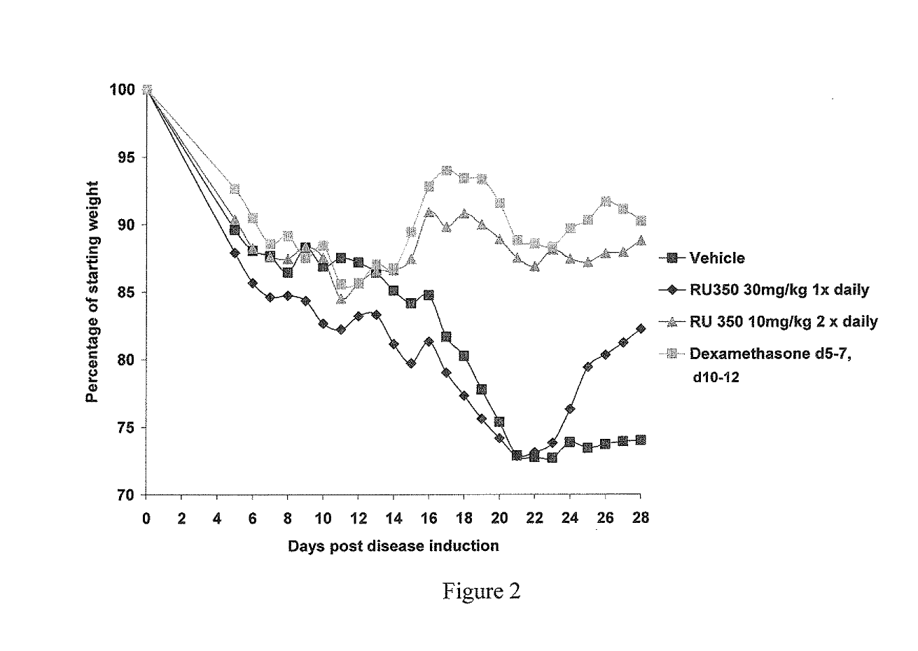

The compound has chiral centres at the 3 and 11b carbon atoms and hence can, theoretically, exist in a total of four isomeric forms, as shown in FIG. 2.

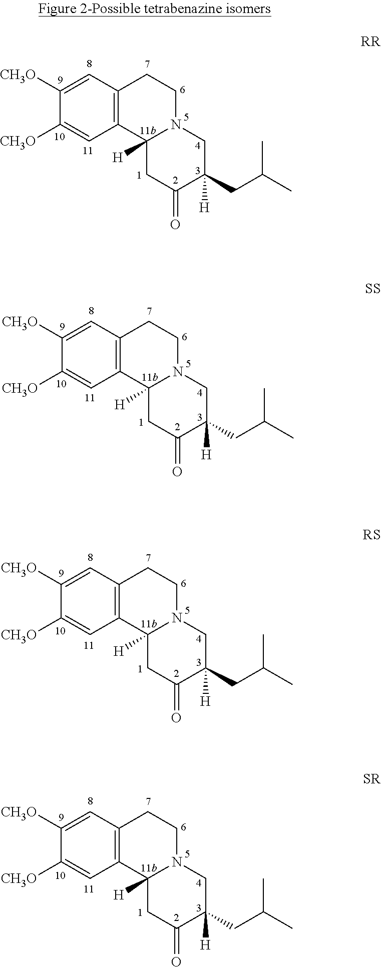

In FIG. 2, the stereochemistry of each isomer is defined using the “R and S” nomenclature developed by Cahn, Ingold and Prelog, see Advanced Organic Chemistry by Jerry March, 4^{th} Edition, John Wiley & Sons, New York, 1992, pages 109-114. In FIG. 2 and elsewhere in this patent application, the designations “R” or “S” are given in the order of the position numbers of the carbon atoms. Thus, for example, RS is a shorthand notation for 3R,11bS. Similarly, when three chiral centres are present, as in the dihydrotetrabenazines described below, the designations “R” or “S” are listed in the order of the carbon atoms 2, 3 and 11b. Thus, the 2S,3R,11bR isomer is referred to in short hand form as SRR and so on.

Commercially available tetrabenazine is a racemic mixture of the RR and SS isomers and it would appear that the RR and SS isomers (hereinafter referred to individually or collectively as trans-tetrabenazine because the hydrogen atoms at the 3 and 11b positions have a trans relative orientation) are the most thermodynamically stable isomers.

Tetrabenazine has somewhat poor and variable bioavailability. It is extensively metabolised by first-pass metabolism, and little or no unchanged tetrabenazine is typically detected in the urine. The major metabolite is dihydrotetrabenazine (Chemical name: 2-hydroxy-3-(2-methylpropyl)-1,3,4,6,7,11b-hexahydro-9,10-dimethoxy-benzo(a)quinolizine) which is formed by reduction of the 2-keto group in tetrabenazine, and is believed to be primarily responsible for the activity of the drug (see Mehvar et al., Drug Metab. Disp, 15, 250-255 (1987) and J. Pharm. Sci., 76, No. 6, 461-465 (1987)), and Roberts et al., Eur. J. Clin. Pharmacol., 29: 703-708 (1986).

Four dihydrotetrabenazine isomers have previously been identified that are derived from the more stable RR and SS isomers of the parent tetrabenazine and have a trans relative orientation between the hydrogen atoms at the 3 and 11b positions) (see Kilbourn et al., Chirality, 9:59-62 (1997) and Brossi et al., Helv. Chim. Acta., vol. XLI, No. 193, pp 1793-1806 (1958). The four isomers, referred to collectively hereinafter as the trans-dihydrotetrabenazines, are (+)-α-dihydrotetrabenazine, (−)-α-dihydrotetrabenazine, (+)-β-dihydrotetrabenazine and (−)-β-dihydrotetrabenazine. The structures of the four trans dihydrotetrabenazine isomers are considered to be as shown in FIG. 3.

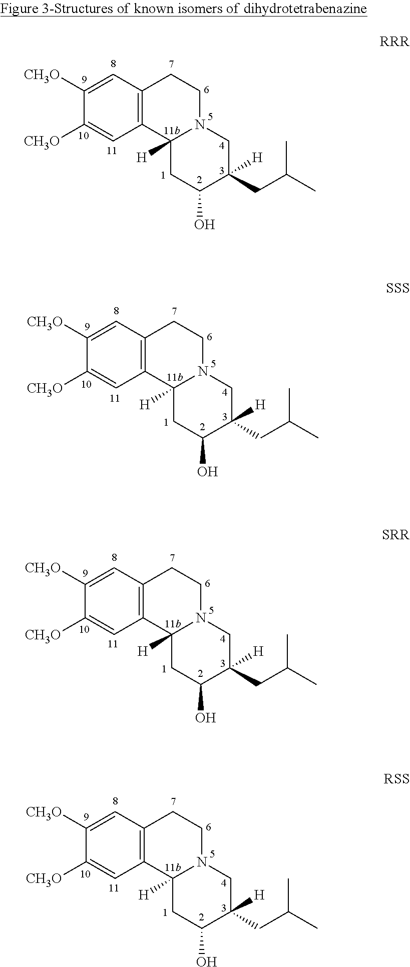

Kilbourn et al., (see Eur. J. Pharmacol., 278:249-252 (1995) and Med. Chem. Res., 5:113-126 (1994)) investigated the specific binding of individual radio-labelled dihydrotetrabenazine isomers in the conscious rat brain. They found that the (+)-α-[¹¹C]dihydrotetrabenazine (2R,3R,11bR) isomer accumulated in regions of the brain associated with higher concentrations of the neuronal membrane dopamine transporter (DAT) and the vesicular monoamine transporter (VMAT2). However, the essentially inactive (−)-α-[¹¹C]dihydrotetrabenazine isomer was almost uniformly distributed in the brain, suggesting that specific binding to DAT and VMAT2 was not occurring. The in vivo studies correlated with in vitro studies which demonstrated that the (+)-α-[¹¹C]dihydrotetrabenazine isomer exhibits a Kᵢ for [³H]methoxytetrabenazine >2000-fold higher than the Kᵢ for the (+)-α-[¹¹C]drotetrabenazine isomer.

International patent application number PCT/GB2005/000464 (Publication number WO 2005/077946) discloses the preparation and use of pharmaceutical dihydrotetrabenazine isomers derived from the unstable RS and SR isomers (hereinafter referred to individually or collectively as cis-tetrabenazine because the hydrogen atoms at the 3 and 11b positions have a cis relative orientation) of tetrabenazine.

The four cis-dihydrotetrabenazine isomers are:

(a) the 2S,3S,11bR isomer of 3,11b-cis-dihydrotetrabenazine having the formula (Ia):

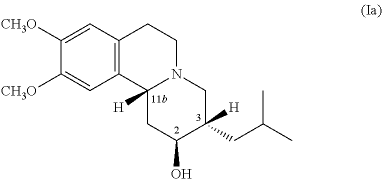

(b) the 2R,3R,11bS isomer of 3,11b-cis-dihydrotetrabenazine having the formula (Ib):

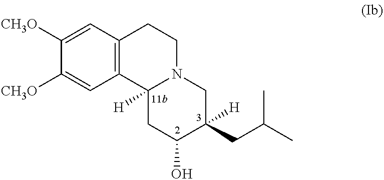

(c) the 2R,3S,11bR isomer of 3,11b-cis-dihydrotetrabenazine having the formula (Ic):

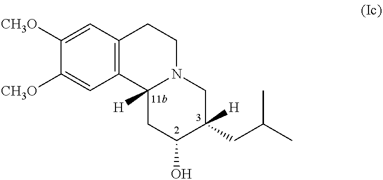

and
(d) the 2S,3R,11bS isomer of 3,11b-cis-dihydrotetrabenazine having the formula (Id):

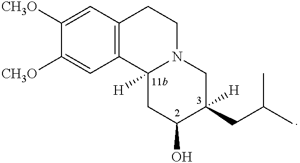

Our application WO 2007/017643 discloses the use of the 3,11b-cis-dihydrotetrabenazines as anti-inflammatory agents but does not disclose the use of the compounds in the treatment of multiple sclerosis.

### SUMMARY OF THE INVENTION

It has now been found that the 2S,3S,11bR isomer of 3,11b-cis-dihydrotetra-benazine (formula (Ia) above) inhibits the development of autoimmune encephalomyelitis, an established model of multiple sclerosis. On the basis of the experimental results obtained to date, it is envisaged that the compound of formula (Ia) will be useful for treating multiple sclerosis in humans.

Accordingly, in a first aspect, the invention provides a compound for use in treating multiple sclerosis, wherein the compound is a 3,11b-cis-dihydrotetrabenazine of the formula (Ia):

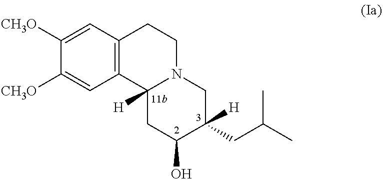

or a pharmaceutically acceptable salt thereof.

In another aspect, the invention provides a compound for use in treating an autoimmune myelitis wherein the compound is a 3,11b-cis-dihydrotetrabenazine of the formula (Ia) or a pharmaceutically acceptable salt thereof as defined herein.

In further aspects, the invention provides:
The use of a compound which is a 3,11b-cis-dihydrotetrabenazine of the formula (Ia) or a pharmaceutically acceptable salt thereof as defined herein for the manufacture of a medicament for treating multiple sclerosis.
The use of a compound which is a 3,11b-cis-dihydrotetrabenazine of the formula (Ia) or a pharmaceutically acceptable salt thereof as defined herein for the manufacture of a medicament for treating an autoimmune myelitis.
A method of treating multiple sclerosis which method comprises administering to a patient in need thereof, an effective treatment amount of a 3,11b-cis-dihydrotetrabenazine of the formula (Ia) or a pharmaceutically acceptable salt thereof.
A method of treating an autoimmune myelitis which method comprises administering to a patient in need thereof, an effective treatment amount of a 3, 11b-cis-dihydrotetrabenazine of the formula (Ia) or a pharmaceutically acceptable salt thereof.

The 3,11b-cis-dihydrotetrabenazine of the formula (Ia) may be referred to herein for convenience by the synonyms “the compound of formula (Ia)” or “the compound of the invention” or “the isomer of the invention” or “Isomer B”.

The compound of formula (Ia) may be used in substantially pure form, for example at an isomeric purity of greater than 90%, typically greater than 95% and more preferably greater than 98%.

The term “isomeric purity” in the present context refers to the amount of the compound of formula (Ia) relative to the total amount or concentration of dihydrotetrabenazine of all isomeric forms. For example, if 90% of the total dihydrotetrabenazine present in the composition is the compound of formula (Ia), then the isomeric purity is 90%.

The 3,11b-cis-dihydrotetrabenazine compound of formula (Ia) used in the invention may be in the form of a composition which is substantially free of 3,11b-trans-dihydrotetrabenazine, preferably containing less than 5% of 3,11b-trans-dihydrotetrabenazine, more preferably less than 3% of 3,11b-trans-dihydrotetrabenazine, and most preferably less than 1% of 3,11b-trans-dihydrotetrabenazine.

The term “3,11b-cis-” as used herein means that the hydrogen atoms at the 3- and 11b-positions of the dihydrotetrabenazine structure are in the cis relative orientation

The 3,11b-cis-dihydrotetrabenazine compound of formula (Ia) may be presented in a substantially enantiomerically pure form or as mixtures with other enantiomers of The 3,11b-cis-dihydrotetrabenazine.

The terms “enantiomeric purity” and “enantiomerically pure” in the present context refer to the amount of a given enantiomer of 3,11b-cis-dihydrotetrabenazine present relative to the total amount or concentration of dihydrotetrabenazine of all enantiomeric and isomeric forms. For example, if 90% of the total dihydrotetrabenazine present in the composition is in the form of a single enantiomer, then the enantiomeric purity is 90%.

By way of example, in each aspect and embodiment of the invention, the 3,11b-cis-dihydrotetrabenazine compound of formula (Ia) may be present in an enantiomeric purity of at least 55% (e.g. at least 60%, 65%, 70%, 75%, 80%, 85%, 90%, 95%, 97%, 98%, 99%, 99.5% or 100%). Most preferably, the 3,11b-cis-dihydrotetrabenazine compound of formula (Ia) is substantially free of other dihydrotetrabenazine isomers.

### Pharmaceutically Acceptable Salts

Unless the context requires otherwise, a reference in this application to the 3,11b-cis-dihydrotetrabenazine compound of formula (Ia), or synonyms thereof, includes within its scope not only the free base of the compound but also its salts, and in particular acid addition salts.

Particular acids from which the acid addition salts are formed include acids having a pKa value of less than 3.5 and more usually less than 3. For example, the acid addition salts can be formed from an acid having a pKa in the range from +3.5 to −3.5.

Preferred acid addition salts include those formed with sulphonic acids such as methanesulphonic acid, ethanesulphonic acid, benzene sulphonic acid, toluene sulphonic acid, camphor sulphonic acid and naphthalene sulphonic acid.

One particular acid from which acid addition salts may be formed is methanesulphonic acid.

Acid addition salts can be prepared by the methods described herein or conventional chemical methods such as the methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free base form of the compound with the appropriate base or acid in water or in an organic solvent, or in a mixture of the two; generally, nonaqueous media such as ether, ethyl acetate, ethanol, isopropanol, or acetonitrile are used.

The salts are typically pharmaceutically acceptable salts. However, salts that are not pharmaceutically acceptable may also be prepared as intermediate forms which may then be converted into pharmaceutically acceptable salts. Such non-pharmaceutically acceptable salt forms also form part of the invention.

### Methods for the Preparation of the Compound of Formula (Ia)

The dihydrotetrabenazine of the invention can be prepared by a process comprising the reaction of a compound of the formula (II):

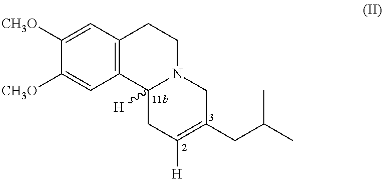

with a reagent or reagents suitable for hydrating the 2,3-double bond in the compound of formula (II) and thereafter where required separating and isolating the desired dihydrotetrabenazine isomer form.

The hydration of the 2,3-double bond can be carried out by hydroboration using a borane reagent such as diborane or a borane-ether (e.g. borane-tetrahydrofuran (THF)) to give an intermediate alkyl borane adduct followed by oxidation of the alkyl borane adduct and hydrolysis in the presence of a base. The hydroboration is typically carried out in a dry polar non-protic solvent such as an ether (e.g. THF), usually at a non-elevated temperature, for example room temperature. The borane-alkene adduct is typically oxidised with an oxidising agent such as hydrogen peroxide in the presence of a base providing a source of hydroxide ions, such as ammonium hydroxide or an alkali metal hydroxide, e.g. potassium hydroxide or sodium hydroxide. The hydroboration-oxidation-hydrolysis sequence of reactions of Process A typically provides dihydrotetrabenazine isomers in which the hydrogen atoms at the 2- and 3-positions have a trans relative orientation.

Compounds of the formula (II) can be prepared by reduction of tetrabenazine to give a dihydrotetrabenazine followed by dehydration of the dihydrotetrabenazine. Reduction of the tetrabenazine can be accomplished using an aluminium hydride reagent such as lithium aluminium hydride, or a borohydride reagent such as sodium borohydride, potassium borohydride or a borohydride derivative, for example an alkyl borohydride such as lithium tri-sec-butyl borohydride.

Alternatively, the reduction step can be effected using catalytic hydrogenation, for example over a Raney nickel or platinum oxide catalyst. Suitable conditions for performing the reduction step are described in more detail below or can be found in U.S. Pat. No. 2,843,591 (Hoffmann-La Roche) and Brossi et al., Helv. Chim. Acta., vol. XLI, No. 193, pp 1793-1806 (1958).

Because the tetrabenazine used as the starting material for the reduction reaction is typically a mixture of the RR and SS isomers (i.e. trans-tetrabenazine), the dihydrotetrabenazine formed by the reduction step will have the same trans configuration about the 3- and 11b positions and will take the form of one or more of the known dihydrotetrabenazine isomers shown in FIG. 3 above. Thus the process may involve taking the known isomers of dihydrotetrabenazine, dehydrating them to form the alkene (II) and then “rehydrating” the alkene (II) using conditions that give the required cis dihydrotetrabenazine isomer of the invention.

Dehydration of the dihydrotetrabenazine to the alkene (II) can be carried out using a variety of standard conditions for dehydrating alcohols to form alkenes, see for example J. March (idem) pages 389-390 and references therein. Examples of such conditions include the use of phosphorus-based dehydrating agents such as phosphorus halides or phosphorus oxyhalides, e.g. POCl₃ and PCl₅. As an alternative to direct dehydration, the hydroxyl group of the dihydrotetrabenazine can be converted to a leaving group L such as halogen (e.g. chlorine or bromine) and then subjected to conditions (e.g. the presence of a base) for eliminating H-L. Conversion of the hydroxyl group to a halide can be achieved using methods well known to the skilled chemist, for example by reaction with carbon tetrachloride or carbon tetrabromide in the presence of a trialkyl or triaryl phosphine such as triphenyl phosphine or tributyl phosphine.

The tetrabenazine used as the starting material for the reduction to give the dihydrotetrabenazine can be obtained commercially or can be synthesised by the method described in U.S. Pat. No. 2,830,993 (Hoffmann-La Roche).

When the starting materials for the process above are mixtures of enantiomers, then the products of the processes will typically be pairs of enantiomers, for example racemic mixtures, possibly together with diastereoisomeric impurities. Unwanted diastereoisomers can be removed by techniques such as chromatography (e.g. HPLC) and the individual enantiomers can be separated by a variety of methods known to the skilled chemist. For example, they can be separated by means of:

(i) chiral chromatography (chromatography on a chiral support); or

(ii) forming a salt with an optically pure chiral acid, separating the salts of the two diastereoisomers by fractional crystallisation and then releasing the dihydrotetrabenazine from the salt; or

(iii) forming a derivative (such as an ester) with an optically pure chiral derivatising agent (e.g. esterifying agent), separating the resulting epimers (e.g. by chromatography) and then converting the derivative to the dihydrotetrabenazine.

One method of separating pairs of enantiomers obtained from Process, and which has been found to be particularly effective, is to esterify the hydroxyl group of the dihydrotetrabenazine with an optically active form of Mosher's acid, such as the R (+) isomer shown below, or an active form thereof:

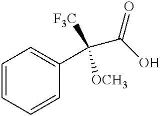

The resulting esters of the two enantiomers of the dihydrobenazine can then be separated by chromatography (e.g. HPLC) and the separated esters hydrolysed to give the individual dihydrobenazine isomers using a base such as an alkali metal hydroxide (e.g. NaOH) in a polar solvent such as methanol.

As an alternative to using mixtures of enantiomers as the starting materials in the process and then carrying out separation of enantiomers subsequently, the process can each be carried out on single enantiomer starting materials leading to products in which a single enantiomer predominates. Single enantiomers of the alkene (II) can be prepared by subjecting RR/SS tetrabenazine to a stereoselective reduction using lithium tri-sec-butyl borohydride to give a mixture of SRR and RSS enantiomers of dihydrotetrabenazine, separating the enantiomers (e.g. by fractional crystallisation) and then dehydrating a separated single enantiomer of dihydrotetrabenazine to give predominantly or exclusively a single enantiomer of the compound of formula (II).

The process is illustrated in more detail below in Scheme 1 below.

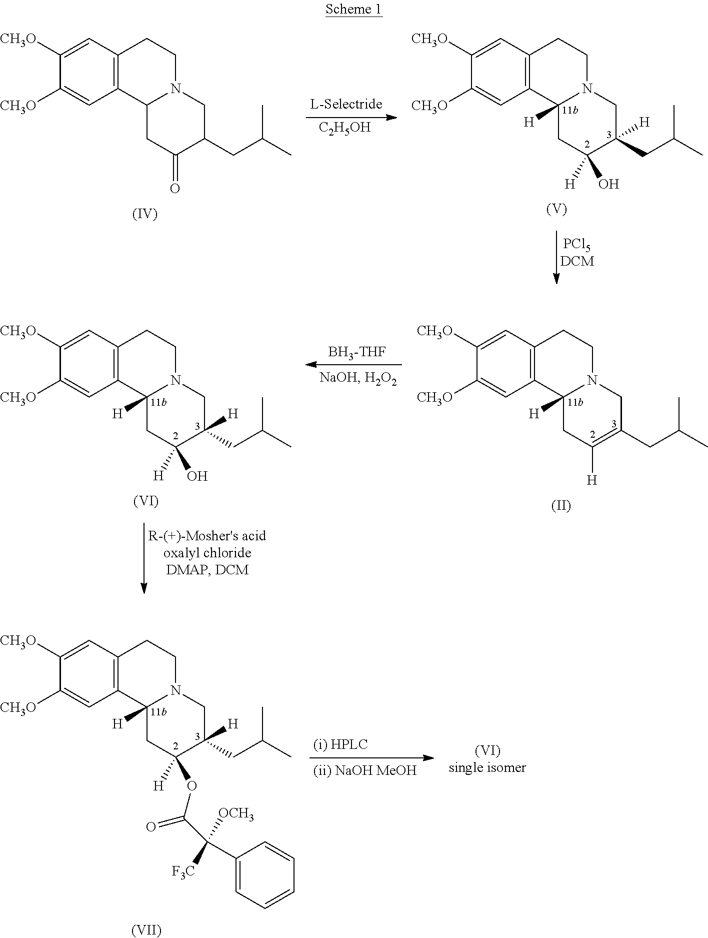

Scheme 1 illustrates the preparation of individual dihydrotetrabenazine isomers having the 2S,3S,11bR and 2R,3R,11bS configurations in which the hydrogen atoms attached to the 2- and 3-positions are arranged in a trans relative orientation.

The starting point for the sequence of reactions in Scheme 1 is commercially available tetrabenazine (IV) which is a racemic mixture of the RR and SS optical isomers of tetrabenazine. In each of the RR and SS isomers, the hydrogen atoms at the 3- and 11b-positions are arranged in a trans relative orientation. As an alternative to using the commercially available compound, tetrabenazine can be synthesised according to the procedure described in U.S. Pat. No. 2,830,993 (see in particular example 11).

The racemic mixture of RR and SS tetrabenazine is reduced using the borohydride reducing agent lithium tri-sec-butyl borohydride (“L-Selectride”) to give a mixture of the known 2S,3R,11bR and 2R,3S,11bS isomers (V) of dihydrotetrabenazine, of which only the 2S,3R,11bR isomer is shown for simplicity. By using the more sterically demanding L-Selectride as the borohydride reducing agent rather than sodium borohydride, formation of the RRR and SSS isomers of dihydro-tetrabenazine is minimised or suppressed.

The dihydrotetrabenazine isomers (V) are reacted with a dehydrating agent such as phosphorus pentachloride in a non-protic solvent such as a chlorinated hydrocarbon (for example chloroform or dichloromethane, preferably dichloromethane) to form the unsaturated compound (II) as a pair of enantiomers, only the R-enantiomer of which is shown in the Scheme. The dehydration reaction is typically carried out at a temperature lower than room temperature, for example at around 0-5° C.

The unsaturated compound (II) is then subjected to a stereoselective re-hydration to generate the dihydrotetrabenazine (VI) and its mirror image or antipode (not shown) in which the hydrogen atoms at the 3- and 11b-positions are arranged in a cis relative orientation and the hydrogen atoms at the 2- and 3-positions are arranged in a trans relative orientation. The stereoselective rehydration is accomplished by a hydroboration procedure using borane-THF in tetrahydrofuran (THF) to form an intermediate borane complex (not shown) which is then oxidised with hydrogen peroxide in the presence of a base such as sodium hydroxide.

An initial purification step may then be carried out (e.g. by HPLC) to give the product (V) of the rehydration reaction sequence as a mixture of the 2S,3S,11bR and 2R,3R,11bS isomers of which only the 2S,3S,11bR isomer is shown in the Scheme. In order to separate the isomers, the mixture is treated with R (+) Mosher's acid, in the presence of oxalyl chloride and dimethylaminopyridine (DMAP) in dichloromethane to give a pair of diastereoisomeric esters (VII) (of which only one diastereoisomer is shown) which can then be separated using HPLC. The individual esters can then be hydrolysed using an alkali metal hydroxide such as sodium hydroxide to give a single isomer (VI).

In a variation of the sequence of steps shown in Scheme 1, following the reduction of RR/SS tetrabenazine, the resulting mixture of enantiomers of the dihydrotetrabenazine (V) can be separated to give the individual enantiomers. Separation can be carried out by forming a salt with a chiral acid such as (+) or (−) camphorsulphonic acid, separating the resulting diastereoisomers by fractional crystallisation to give a salt of a single enantiomer and then releasing the free base from the salt.

The separated dihydrotetrabenazine enantiomer can be dehydrated to give a single enantiomer of the alkene (II). Subsequent rehydration of the alkene (II) will then give predominantly or exclusively a single enantiomer of the cis-dihydro-tetrabenazine (VI). An advantage of this variation is that it does not involve the formation of Mosher's acid esters and therefore avoids the chromatographic separation typically used to separate Mosher's acid esters.

### Biological Properties and Therapeutic Uses

The compound of formula (Ia) has been tested in an experimental autoimmune encephalomyelitis model of multiple sclerosis and has been found to give levels of protection similar to that provided by treatment with steroids. On the basis of this evidence, it is envisaged that the compound of formula (Ia) will be useful in the treatment of multiple sclerosis in humans.

The terms “treating” and “treatment” as used herein in the context of multiple sclerosis include any one or more of:
halting the progression of the disease;
slowing the progression of the disease;
modifying the progression of the disease;
providing symptomatic relief, e.g. by eliminating or reducing the severity of one or more symptoms;
extending periods of remission;
preventing relapses;
reducing the severity of relapses; and
preventing or slowing the progression from an initial period of relapsing-remitting MS to secondary progressive MS.

Symptoms of multiple sclerosis that may be eliminated or reduced in severity in accordance with the invention include any one or more symptoms, in any combination, selected from:
weakness and/or numbness in one or more extremities;
tingling of the extremities;
tight band-like sensations around the trunk or limbs;
tremor of one or more extremities;
dragging or poor control of one or both legs;
spastic or ataxic paraparesis;
paralysis of one or more extremities;
hyperactive tendon reflexes;
disappearance of abdominal reflexes;
Lhermitte's sign;
retrobulbar or optic neuritis;
unsteadiness in walking;
problems with balance,
increased muscle fatigue;
brain stem symptoms (diplopia, vertigo, vomiting);
disorders of micturition;
hemiplegia;
trigeminal neuralgia;
other pain syndromes;
nystagmus and ataxia;
cerebellar-type ataxia;
Charcot's triad; diplopia;
bilateral internuclear ophthalmoplegia;
myokymia or paralysis of facial muscles;
deafness;
tinnitus;
unformed auditory hallucinations (because of involvement of cochlear connections);
transient facial anesthesia or of trigeminal neuralgia;
urinary and/or faecal incontinence
bladder dysfunction euphoria;
depression;
fatigue;
dementia;
dull, aching pain in the low back;
sharp, burning, poorly localized pains in a limb;
abrupt attacks of neurologic deficit;
dysarthria and ataxia;
paroxysmal pain and dysesthesia in a limb;
flashing lights;
paroxysmal itching;
tonic seizures;
changes in sensation;
visual problems;
muscle weakness;
difficulties with coordination and speech;
cognitive impairment;
overheating; and
impaired mobility and disability.

The compound may be used in a prophylactic sense during periods of remission in order to prevent or reduce the likelihood or severity of relapses or it may be used to treat patients who are suffering from a relapse. Preferably it is used in a prophylactic sense.

The compound of formula (Ia) will generally be administered to a subject in need of such administration, for example a human patient.

The compound will typically be administered in amounts that are therapeutically or prophylactically useful and which generally are non-toxic. However, in certain situations, the benefits of administering a dihydrotetrabenazine compound of the invention may outweigh the disadvantages of any toxic effects or side effects, in which case it may be considered desirable to administer compounds in amounts that are associated with a degree of toxicity.

A typical daily dose of the compound can be up to 1000 mg per day, for example in the range from 0.01 milligrams to 10 milligrams per kilogram of body weight, more usually from 0.025 milligrams to 5 milligrams per kilogram of body weight, for example up to 3 milligrams per kilogram of bodyweight, and more typically 0.15 milligrams to 5 milligrams per kilogram of bodyweight although higher or lower doses may be administered where required.

By way of example, an initial starting dose of 12.5 mg may be administered 2 to 3 times a day. The dosage can be increased by 12.5 mg a day every 3 to 5 days until the maximal tolerated and effective dose is reached for the individual as determined by the physician. Ultimately, the quantity of compound administered will be commensurate with the nature of the disease or physiological condition being treated and the therapeutic benefits and the presence or absence of side effects produced by a given dosage regimen, and will be at the discretion of the physician.

The compound of the formula (Ia) or a pharmaceutically acceptable salt thereof may be used as the sole therapeutic agent or it may be used in conjunction with other therapeutic agents such as steroids or interferons.

In one general embodiment of the invention, the compound of the formula (Ia) or pharmaceutically acceptable salt thereof is used as the sole therapeutic agent.

### Pharmaceutical Formulations

The compound of formula (Ia) or pharmaceutically acceptable salt thereof is typically administered in the form of a pharmaceutical composition.

The pharmaceutical compositions can be in any form suitable for oral, parenteral, topical, intranasal, intrabronchial, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery.

Pharmaceutical dosage forms suitable for oral administration include tablets, capsules, caplets, pills, lozenges, syrups, solutions, sprays, powders, granules, elixirs and suspensions, sublingual tablets, sprays, wafers or patches and buccal patches.

Pharmaceutical compositions containing the compound of the invention can be formulated in accordance with known techniques, see for example, Remington's Pharmaceutical Sciences, Mack Publishing Company, Easton, Pa., USA.

Thus, tablet compositions can contain a unit dosage of active compound together with an inert diluent or carrier such as a sugar or sugar alcohol, e.g.; lactose, sucrose, sorbitol or mannitol; and/or a non-sugar derived diluent such as sodium carbonate, calcium phosphate, talc, calcium carbonate, or a cellulose or derivative thereof such as methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (e.g.; tablets, capsules etc.) can be coated or un-coated, but typically have a coating, for example a protective film coating (e.g. a wax or varnish) or a release controlling coating. The coating (e.g. a Eudragit™ type polymer) can be designed to release the active component at a desired location within the gastro-intestinal tract. Thus, the coating can be selected so as to degrade under certain pH conditions within the gastrointestinal tract, thereby selectively release the compound in the stomach or in the ileum or duodenum.

Instead of, or in addition to, a coating, the drug can be presented in a solid matrix comprising a release controlling agent, for example a release delaying agent which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract.

Compositions for topical use include ointments, creams, sprays, patches, gels, liquid drops and inserts (for example intraocular inserts). Such compositions can be formulated in accordance with known methods.

Compositions for parenteral administration are typically presented as sterile aqueous or oily solutions or fine suspensions, or may be provided in finely divided sterile powder form for making up extemporaneously with sterile water for injection.

Examples of formulations for rectal or intra-vaginal administration include pessaries and suppositories which may be, for example, formed from a shaped mouldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The compound of the invention will generally be presented in unit dosage form and, as such, will typically contain sufficient compound to provide a desired level of biological activity. For example, a formulation intended for oral administration may contain from 2 milligrams to 200 milligrams of active ingredient, more usually from 10 milligrams to 100 milligrams, for example, 12.5 milligrams, 25 milligrams and 50 milligrams.

The active compound will be administered to a patient in need thereof (for example a human or animal patient) in an amount sufficient to achieve the desired therapeutic effect.

### EXAMPLES

The following non-limiting examples illustrate the synthesis and properties of the compound of the invention.

### Example 1

### Preparation of 2S,3S,11bR and 2R,3R,11bS Isomers of Dihydrotetrabenazine

### 1A. Reduction of RR/SS Tetrabenazine

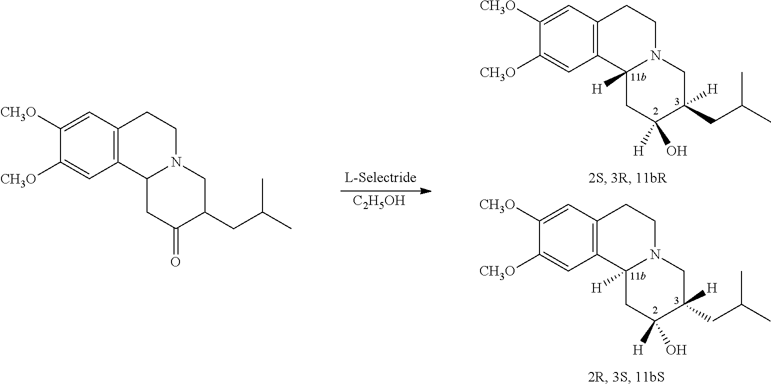

1M L-Selectride in tetrahydrofuran (135 ml, 135 mmol, 2.87 eq.) was added slowly over 30 minutes to a stirred solution of tetrabenazine RR/SS racemate (15 g, 47 mmol) in ethanol (75 ml) and tetrahydrofuran (75 ml) at 0° C. After addition was complete the mixture was stirred at 0° C. for 30 minutes and then allowed to warm to room temperature.

The mixture was poured onto crushed ice (300 g) and water (100 ml) added. The solution was extracted with diethyl ether (2×200 ml) and the combined ethereal extracts washed with water (100 ml) and partly dried over anhydrous potassium carbonate. Drying was completed using anhydrous magnesium sulphate and, after filtration, the solvent was removed at reduced pressure (shielded from the light, bath temperature <20° C.) to afford a pale yellow solid.

The solid was slurried with petroleum ether (30-40° C.) and filtered to afford a white powdery solid (12 g, 80%).

### 1B. Dehydration of Reduced Tetrabenazine

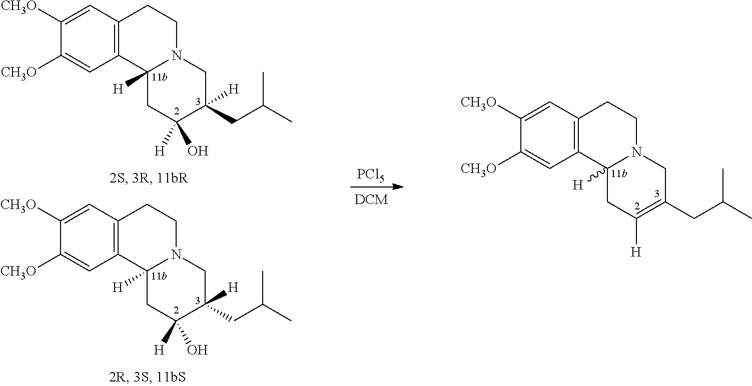

Phosphorous pentachloride (32.8 g, 157.5 mmol, 2.5 eq) was added in portions over 30 minutes to a stirred solution of the reduced tetrabenazine product from Example 1A (20 g, 62.7 mmol) in dichloromethane (200 ml) at 0° C. After the addition was complete, the reaction mixture was stirred at 0° C. for a further 30 minutes and the solution poured slowly into 2M aqueous sodium carbonate solution containing crushed ice (0° C.). Once the initial acid gas evolution had ceased the mixture was basified (ca. pH 12) using solid sodium carbonate.

The alkaline solution was extracted using ethyl acetate (800 ml) and the combined organic extracts dried over anhydrous magnesium sulphate. After filtration the solvent was removed at reduced pressure to afford a brown oil, which was purified by column chromatography (silica, ethyl acetate) to afford the semi-pure alkene as a yellow solid (10.87 g, 58%).

1C. Hydration of the Crude Alkene from Example 1B

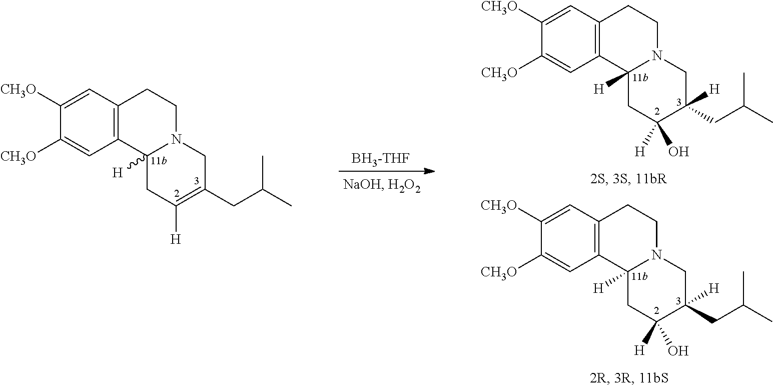

A solution of the crude alkene (10.87 g, 36.11 mmol) from Example 1B in dry THF (52 ml) at room temperature was treated with 1M borane-THF (155.6 ml, 155.6 mmol, 4.30 eq) added in a dropwise manner. The reaction was stirred for 2 hours, water (20 ml) was added and the solution basified to pH 12 with 30% aqueous sodium hydroxide solution.

Aqueous 30% hydrogen peroxide solution (30 ml) was added to the stirred alkaline reaction mixture and the solution was heated to reflux for 1 hour before being allowed to cool. Water (100 ml) was added and the mixture extracted with ethyl acetate (3×250 ml). The organic extracts were combined and dried over anhydrous magnesium sulphate and after filtration the solvent was removed at reduced pressure to afford a yellow oil (9 g).

The oil was purified using preparative HPLC (Column: Lichrospher Si60, 5 μm, 250×21.20 mm, mobile phase: hexane:ethanol:dichloromethane (85:15:5); UV 254 nm, flow: 10 ml min⁻¹) at 350 mg per injection followed by concentration of the fractions of interest under vacuum. The product oil was then dissolved in ether and concentrated once more under vacuum to give the dihydrotetrabenazine racemate shown above as a yellow foam (5.76 g, 50%).

### 1D. Preparation of Mosher's Ester Derivatives

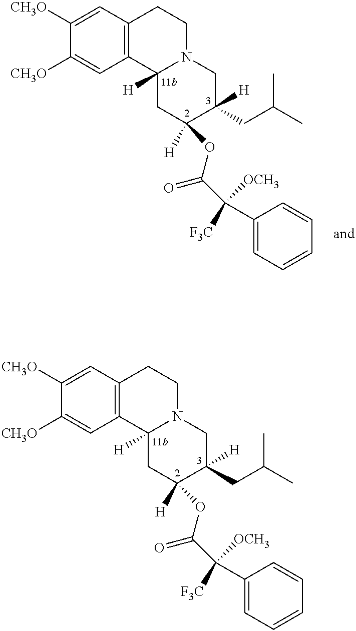

R-(+)-α-methoxy-α-trifluoromethylphenyl acetic acid (5 g, 21.35 mmol), oxalyl chloride (2.02 ml) and DMF (0.16 ml) were added to anhydrous dichloromethane (50 ml) and the solution was stirred at room temperature for 45 minutes. The solution was concentrated under reduced pressure and the residue was taken up in anhydrous dichloromethane (50 ml) once more. The resulting solution was cooled using an ice-water bath and dimethylaminopyridine (3.83 g, 31.34 mmol) was added followed by a pre-dried solution (over 4A sieves) in anhydrous dichloromethane of the solid product of Example 1C (5 g, 15.6 mmol). After stirring at room temperature for 45 minutes, water (234 ml) was added and the mixture extracted with ether (2×200 ml). The ether extract was dried over anhydrous magnesium sulphate, passed through a pad of silica and the product eluted using ether.

The collected ether eluate was concentrated under reduced pressure to afford an oil which was purified using column chromatography (silica, hexane:ether (10:1)).

Evaporation of the collected column fractions of interest and removal of the solvent at reduced pressure gave a solid which was further purified using column chromatography (silica, hexane:ethyl acetate (1:1)) to give three main components which were partially resolved into Mosher's ester peaks 1 and 2.

Preparative HPLC of the three components (Column: 2×Lichrospher Si60, 5 μm, 250×21.20 mm, mobile phase: hexane:isopropanol (97:3), UV 254 nm; flow: 10 ml min⁻¹) at 300 mg loading followed by concentration of the fractions of interest under vacuum gave the pure Mosher's ester derivatives

### Peak 1 (3.89 g, 46.5%)

### Peak 2 (2.78 g, 33%)

The fractions corresponding to the two peaks were subjected to hydrolysis to liberate the individual dihydrotetrabenazine isomers identified and characterised as Isomers A and B. Isomers A and B are each believed to have one of the following structures

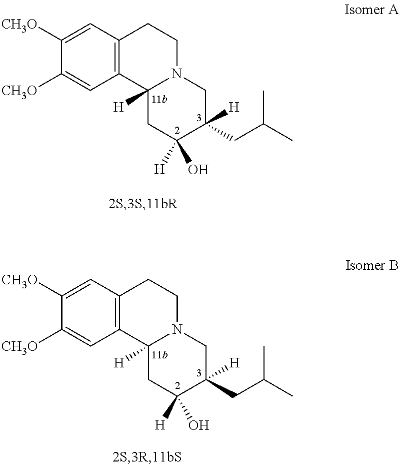

More specifically, Isomer B is believed to have the 2S, 3S, 11bR absolute configuration on the basis of the X-ray crystallography experiments described in Example 4 below.

### 1E. Hydrolysis of Peak 1 to Give Isomer A

Aqueous 20% sodium hydroxide solution (87.5 ml) was added to a solution of Mosher's ester peak 1 (3.89 g, 7.27 mmol) in methanol (260 ml) and the mixture stirred and heated to reflux for 150 minutes. After cooling to room temperature water (200 ml) was added and the solution extracted with ether (600 ml), dried over anhydrous magnesium sulphate and after filtration, concentrated under reduced pressure.

The residue was dissolved using ethyl acetate (200 ml), the solution washed with water (2×50 ml), the organic phase dried over anhydrous magnesium sulphate and after filtration, concentrated under reduced pressure to give a yellow foam. This material was purified by column chromatography (silica, gradient elution of ethyl acetate:hexane (1:1) to ethyl acetate). The fractions of interest were combined and the solvent removed at reduced pressure. The residue was taken up in ether and the solvent removed at reduced pressure once more to give Isomer A as an off-white foam (1.1 g, 47%).

Isomer A, which is believed to have the 2R,3R,11bS configuration (the absolute stereochemistry was not determined), was characterized by ¹H-NMR, ¹³C-NMR, IR, mass spectrometry, chiral HPLC and ORD. The IR, NMR and MS data for isomer A are set out in Table 1 and the Chiral HPLC and ORD data are set out in Table 3.

### 1F. Hydrolysis of Peak 2 to Give Isomer B

Aqueous 20% sodium hydroxide solution (62.5 ml) was added to a solution of Mosher's ester peak 2 (2.78 g, 5.19 mmol) in methanol (185 ml) and the mixture stirred and heated to reflux for 150 minutes. After cooling to room temperature water (142 ml) was added and the solution extracted with ether (440 ml), dried over anhydrous magnesium sulphate and after filtration, concentrated under reduced pressure.

The residue was dissolved using ethyl acetate (200 ml), the solution washed with water (2×50 ml), the organic phase dried over anhydrous magnesium sulphate and after filtration, concentrated under reduced pressure. Petroleum ether (30-40° C.) was added to the residue and the solution concentrated under vacuum once more to give Isomer B as a white foam (1.34 g, 81%).

Isomer B, which is believed to have the 2S,3S,11bR configuration, was characterized by ¹H-NMR, ¹³C-NMR, IR, mass spectrometry, chiral HPLC, ORD and X-ray crystallography. The IR, NMR and MS data for Isomer B are set out in Table 1 and the Chiral HPLC and ORD data are set out in Table 2. The X-ray crystallography data are set out in Example 3.

In Table 1, the infra red spectra were determined using the KBr disc method. The ¹H NMR spectra were carried out on solutions in deuterated chloroform using a Varian Gemini NMR spectrometer (200 MHz.). The ¹³C NMR spectra were carried out on solutions in deuterated chloroform using a Varian Gemini NMR spectrometer (50 MHz). The mass spectra were obtained using a Micromass Platform II (ES⁺ conditions) spectrometer. In Table 2, the Optical Rotatory Dispersion figures were obtained using an Optical Activity PolAAr 2001 instrument in methanol solution at 24° C. The HPLC retention time measurements were carried out using an HP1050 HPLC chromatograph with UV detection.

TABLE 1
Spectroscopic Data

| | ¹H-NMR | ¹³C-NMR | IR | Mass |
|---|---|---|---|---|
| | spectrum | spectrum | Spectrum | Spectrum |
| Dihydrotetrabenazine isomer | (CDCl₃) | (CDCl₃) | (KBr solid) | (ES⁺) |
| Isomers A and B 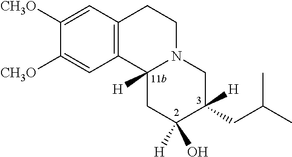 2S,3S,11bR OR 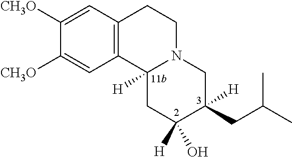 2R,3R,11bS | 6.67 δ 1H (s); 6.57 δ 1H (s); 3.84 δ 6H (s); 3.55 δ 1H (br. d); 3.08 δ 1H (m); 2.79 δ 2H (m); 2.55 δ 3H (m); 2.17 δ 1H (m); 1.72 δ 6H (m); 1.02 δ 1H (m); 0.88 δ 6H (t) | 147.7 δ; 147.6 δ; 130.5 δ; 127.6 δ; 112.1 δ; 108.4 δ; 70.5 δ; 57.5 δ; 56.5 δ; 56.3 δ; 54.8 δ; 53.2 δ; 40.4 δ; 40.1 δ; 36.0 δ; 28.8 δ; 26.2 δ; 23.7 δ; 22.9 δ | 2950 cm⁻¹; 2928 cm⁻¹; 2868 cm⁻¹; 2834 cm⁻¹; 1610 cm⁻¹; 1511 cm⁻¹; 1464 cm⁻¹; 1364 cm⁻¹; 1324 cm⁻¹; 1258 cm⁻¹; 1223 cm⁻¹; 1208 cm⁻¹; 1144 cm⁻¹; 1045 cm⁻¹; 1006 cm⁻¹; 870 cm⁻¹; 785 cm⁻¹; 764 cm⁻¹; | MH⁺ 320 |

TABLE 2
Chromatography and ORD Data

| | | ORD |
|---|---|---|
| Dihydrotetrabenazine isomer | Chiral HPLC Methods and Retention Times | (MeOH, 21° C.) |
| Isomers A and B | Column: | Isomer A |
| | Chirex (S)-VAL, (R)-NEA, 250 × 4.6 mm | [α_{D}] −114.6° |
| | Mobile phase: Hexane:1,2-dichloroethane:ethanol (36:62:2) | |
| 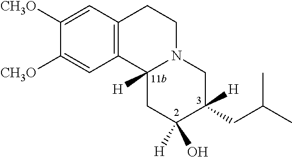 2S,3S,11bR OR 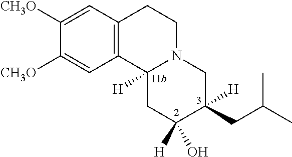 2R,3R,11bS | Flow: 1.0 ml min⁻¹ UV: 254 nm Retention times: Isomer A 16.6 min Isomer B 15.3 min | Isomer B [α_{D}] +123° |

### Example 2

### Alternative Method of Preparation of Isomer B and Preparation of Mesylate Salt 2A. Reduction of RR/SS Tetrabenazine

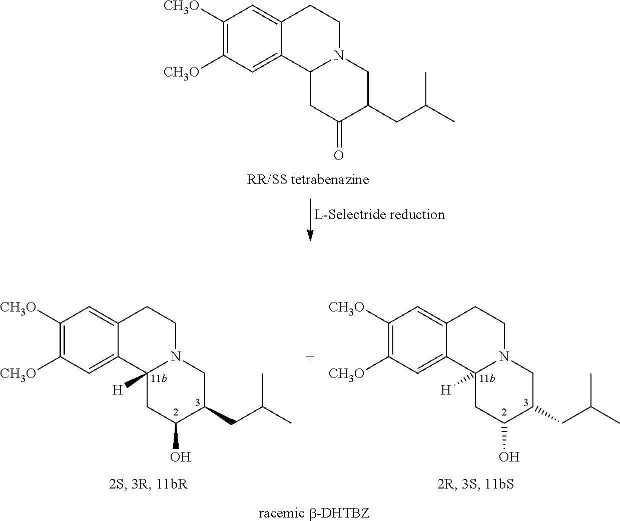

1M L-Selectride in tetrahydrofuran (52 ml, 52 mmol, 1.1 eq) was added slowly over 30 minutes to a cooled (ice bath), stirred solution of tetrabenazine racemate (15 g, 47 mmol) in tetrahydrofuran (56 ml). After the addition was complete, the mixture was allowed to warm to room temperature and stirred for a further six hours. TLC analysis (silica, ethyl acetate) showed only very minor amounts of starting material remained.

The mixture was poured on to a stirred mixture of crushed ice (112 g), water (56 ml) and glacial acetic acid (12.2 g). The resulting yellow solution was washed with ether (2×50 ml) and basified by the slow addition of solid sodium carbonate (ca. 13 g). Pet-ether (30-40° C.) (56 ml) was added to the mixture with stirring and the crude β-DHTBZ was collected as a white solid by filtration.

The crude solid was dissolved in dichloromethane (ca. 150 ml) and the resulting solution washed with water (40 ml), dried using anhydrous magnesium sulphate, filtered and concentrated at reduced pressure to ca. 40 ml. A thick suspension of white solid was formed. Pet-ether (30-40° C.) (56 ml) was added and the suspension was stirred for fifteen minutes at laboratory temperature. The product was collected by filtration and washed on the filter until snow-white using pet-ether (30-40° C.) (40 to 60 ml) before air-drying at room temperature to yield β-DHTBZ (10.1 g, 67%) as a white solid. TLC analysis (silica, ethyl acetate) showed only one component.

### 2B. Preparation and Fractional Crystallisation of the Camphorsulphonic Acid Salt of Racemic β-DHTBZ

The product of Example 3A and 1 equivalent of (S)-(+)-Camphor-10-sulphonic acid were dissolved with heating in the minimum amount of methanol. The resulting solution was allowed to cool and then diluted slowly with ether until formation of the resulting solid precipitation was complete. The resulting white crystalline solid was collected by filtration and washed with ether before drying.

The camphorsulphonic acid salt of (10 g) was dissolved in a mixture of hot absolute ethanol (170 ml) and methanol (30 ml). The resulting solution was stirred and allowed to cool. After two hours the precipitate formed was collected by filtration as a white crystalline solid (2.9 g). A sample of the crystalline material was shaken in a separating funnel with excess saturated aqueous sodium carbonate and dichloromethane. The organic phase was separated, dried over anhydrous magnesium sulphate, filtered and concentrated at reduced pressure. The residue was triturated using pet-ether (30-40° C.) and the organic solution concentrated once more. Chiral HPLC analysis of the salt using a Chirex (S)-VAL and (R)—NEA 250×4.6 mm column, and a hexane:ethanol (98:2) eluent at a flow rate of 1 ml/minute showed that the isolated β-DHTBZ was enriched in one enantiomer (e.e. ca. 80%).

The enriched camphorsulphonic acid salt (14 g) was dissolved in hot absolute ethanol (140 ml) and propan-2-ol (420 ml) was added. The resulting solution was stirred and a precipitate began to form within one minute. The mixture was allowed to cool to room temperature and stirred for one hour. The precipitate formed was collected by filtration, washed with ether and dried to give a white crystalline solid (12 g).

The crystalline material was shaken in a separating funnel with excess saturated aqueous sodium carbonate and dichloromethane. The organic phase was separated, dried over anhydrous magnesium sulphate, filtered and concentrated at reduced pressure. The residue was triturated using pet-ether (30-40° C.) and the organic solution concentrated once more to yield (after drying in vacuo.) (+)-β-DHTBZ (6.6 g, ORD+107.8°). The isolated enantiomer has e.e. >97%.

### 2C. Preparation of Isomer B

A solution of phosphorus pentachloride (4.5 g, 21.6 mmol, 1.05 eq) in dichloromethane (55 ml) was added steadily over ten minutes to a stirred, cooled (ice-water bath) solution of the product of Example 3B (6.6 g, 20.6 mmol) in dichloromethane (90 ml). When the addition was complete, the resulting yellow solution was stirred for a further ten minutes before pouring on to a rapidly stirred mixture of sodium carbonate (15 g) in water (90 ml) and crushed ice (90 g). The mixture was stirred for a further 10 minutes and transferred to a separating funnel.

Once the phases had separated, the brown dichloromethane layer was removed, dried over anhydrous magnesium sulphate, filtered and concentrated at reduced pressure to give the crude alkene intermediate as brown oil (ca. 6.7 g). TLC analysis (silica, ethyl acetate) showed that no (+)-β-DHTBZ remained in the crude product.

The crude alkene was taken up (dry nitrogen atmosphere) in anhydrous tetrahydrofuran (40 ml) and a solution of borane in THF (1 M solution, 2.5 eq, 52 ml) was added with stirring over fifteen minutes. The reaction mixture was then stirred at room temperature for two hours. TLC analysis (silica, ethyl acetate) showed that no alkene intermediate remained in the reaction mixture.

A solution of sodium hydroxide (3.7 g) in water (10 ml) was added to the stirring reaction mixture, followed by an aqueous solution of hydrogen peroxide (50%, ca. 7 ml) and the two-phase mixture formed was stirred at reflux for one hour. TLC analysis of the organic phase at this time (silica, ethyl acetate) showed the appearance of a product with Rf as expected for Isomer B. A characteristic non-polar component was also seen.

The reaction mixture was allowed to cool to room temperature and was poured into a separating funnel. The upper organic layer was removed and concentrated under reduced pressure to remove the majority of THF. The residue was taken up in ether (stabilised (BHT), 75 ml), washed with water (40 ml), dried over anhydrous magnesium sulphate, filtered and concentrated under reduced pressure to give a pale yellow oil (8.1 g).

The yellow oil was purified using column chromatography (silica, ethyl acetate:hexane (80:20), increasing to 100% ethyl acetate) and the desired column fractions collected, combined and concentrated at reduced pressure to give a pale oil which was treated with ether (stabilised, 18 ml) and concentrated at reduced pressure to give Isomer B as a pale yellow solid foam (2.2 g).

Chiral HPLC using the conditions set out in Example 2B confirmed that Isomer B had been produced in an enantiomeric excess (e.e.) of greater than 97%.

The optical rotation was measured using a Bellingham Stanley ADP220 polarimeter and gave an [α_{D}] of +123.5°.

### 2D. Preparation of the Mesylate Salt of Isomer B

The methanesulphonate salt of Isomer B was prepared by dissolving a mixture of 1 equivalent of Isomer B from Example 3C and 1 equivalent of methane sulphonic acid in the minimum amount of ethanol and then adding diethyl ether. The resulting white precipitate that formed was collected by filtration and dried in vacuo to give the mesylate salt in a yield of ca. 85% and a purity (by HPLC) of ca. 96%.

### Example 3

### X-Ray Crystallographic Studies on Isomer B

The (S)-(+)-Camphor-10-sulphonic acid salt of Isomer B was prepared and a single crystal was subjected to X-ray crystallographic studies under the following conditions:

Diffractometer: Nonius KappaCCD area detector (t/i scans and OJ scans to fill asymmetric unit).

Cell determination: DirAx (Duisenberg, A. J. M. (1992). J. Appl. Cryst. 25, 92-96.)

Data collection: Collect (Collect: Data collection software, R. Hooft, Nonius B. V, 1998)

Data reduction and cell refinement: Demo (Z. Otwinowski & W. Minor, Methods in Enzymology (1997) Vol. 276: Macromolecular Crystallography, part A, pp. 307-326; C. W. Carter, Jr & R. M. Sweet, Eds., Academic Press).

Absorption correction: Sheldrick, G. M. SADABS—Bruker Nonius area detector scaling and absorption correction—V2.\0

Structure solution: SHELXS97 (G. M. Sheldrick, Acta Cryst. (1990) A46 467-473). Structure refinement: SHELXL97 (G. M. Sheldrick (1997), University of Göttingen, Germany)

Graphics: Cameron—A Molecular Graphics Package (D. M. Watkin, L. Pearce and C. K. Prout, Chemical Crystallography Laboratory, University of Oxford, 1993)

Special details: All hydrogen atoms were placed in idealised positions and refined using a riding model, except those of the NH and OH which were located in the difference map and refined using restraints. Chirality: NI=R, CI2=S, CI3=S, CI5=R, C21=S, C24=R

The results of the studies are set out below in Tables A, B, C, D and E.

In the Tables, the label RUS0350 refers to Isomer B.

TABLE A

| | | |
|---|---|---|
| Identification code Empirical formula Formula weight Temperature Wavelength Crystal system Space group Unit cell dimensions Volume Z | 2005bdy0585 (RUS0350) C₂₉H₄₅NO₇S 551.72 120(2) K 0.71073 Å Orthorhombic P2₁2₁2₁ a = 7.1732(9) Å b = 12.941(2) Å c = 31.025(4) Å 2880.1(7) Å³ 4 | 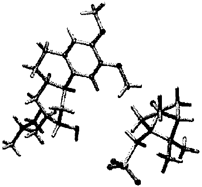 |
| Density (calculated) | 1.272 Mg/m³ | |
| Absorption coefficient | 0.158 mm⁻¹ | |
| F(000) | 1192 | |
| Crystal | Colourless Slab | |
| Crystal size | 0.2 × 0.2 × 0.04 mm³ | |
| θ range for data collection | 3.06 − 27.37° | |
| Index ranges | −8 ≦ h ≦ 9, −16 ≦ k ≦ 16, −36 ≦ l ≦ 39 | |
| Reflections collected | 36802 | |
| Independent reflections | 6326 [Rᵢₙₜ = 0.0863] | |
| Completeness to θ = 27.37° | 97.1% | |
| Absorption correction | Semi-empirical from equivalents | |
| Max. and min. transmission | 0.9937 and 0.9690 | |
| Refinement method | Full-matrix least-squares on F² | |
| Data/restraints/parameters | 6326/1/357 | |
| Goodness-of-fit on F² | 1.042 | |
| Final R indices [F² > 2σ(F²)] | R1 = 0.0498, wR2 = 0.0967 | |
| R indices (all data) | R1 = 0.0901, wR2 = 0.1108 | |
| Absolute structure parameter | 0.04(8) | |
| Extinction coefficient | 0.0059(7) | |
| Largest diff. peak and hole | 0.236 and −0.336 e Å⁻³ | |

TABLE B
Atomic coordinates [×10⁴], equivalent isotropic displacement
parameters [A² × 10³] and site occupancy factors. U_{eq} is
defined as one third of the trace of the orthogonalized U^{ij} tensor.

| Atom | x | y | z | Ueq | S.o.f. |
|---|---|---|---|---|---|
| NI | 4839(3) | 11119(2) | 2180(1) | 24(1) | 1 |
| 01 | 2515(3) | 13171(1) | 349(1) | 31(1) | 1 |
| 02 | 5581(3) | 14030(1) | 598(1) | 32(1) | 1 |
| 03 | 9220(3) | 12834(2) | 2385(1) | 36(1) | 1 |
| CI | 870(4) | 12674(2) | 190(1) | 36(1) | 1 |
| C2 | 3176(3) | 12838(2) | 739(1) | 25(1) | 1 |
| C3 | 2346(4) | 12109(2) | 997(1) | 25(1) | 1 |
| C4 | 3124(3) | 11821(2) | 1395(1) | 24(1) | 1 |
| C5 | 4773(3) | 12276(2) | 1527(1) | 23(1) | 1 |
| C6 | 5629(4) | 13024(2) | 1262(1) | 24(1) | 1 |
| C7 | 4861(4) | 13308(2) | 875(1) | 25(1) | 1 |
| C8 | 7189(4) | 14582(2) | 747(1) | 38(1) | 1 |
| C9 | 2182(3) | 11023(2) | 1673(1) | 28(1) | 1 |
| CI0 | 2759(3) | 11118(2) | 2137(1) | 26(1) | 1 |
| CII | 5366(3) | 11096(2) | 2656(1) | 25(1) | 1 |
| C12 | 7292(4) | 11536(2) | 2747(1) | 25(1) | 1 |
| C13 | 7468(4) | 12663(2) | 2590(1) | 25(1) | 1 |
| C14 | 5988(4) | 12911(2) | 2252(1) | 25(1) | 1 |
| C15 | 5773(4) | 12010(2) | 1943(1) | 24(1) | 1 |
| C16 | 7734(4) | 11477(2) | 3232(1) | 28(1) | 1 |
| C17 | 7752(4) | 10418(2) | 3449(1) | 34(1) | 1 |
| C18 | 9198(6) | 9696(3) | 3249(1) | 65(1) | 1 |
| C19 | 8114(4) | 10562(2) | 3930(1) | 41(1) | 1 |
| C20 | 7509(4) | 8131(2) | 1250(1) | 31(1) | 1 |
| S1 | 7409(1) | 8792(1) | 1754(1) | 27(1) | 1 |
| 04 | 7758(2) | 7965(1) | 2064(1) | 30(1) | 1 |
| 05 | 8831(3) | 9582(2) | 1760(1) | 49(1) | 1 |
| 06 | 5524(2) | 9221(1) | 1798(1) | 32(1) | 1 |
| 07 | 7406(3) | 6932(1) | 498(1) | 48(1) | 1 |
| C21 | 6858(3) | 8622(2) | 830(1) | 25(1) | 1 |
| C22 | 7154(4) | 7851(2) | 459(1) | 30(1) | 1 |
| C23 | 7073(4) | 8450(2) | 40(1) | 32(1) | 1 |
| C24 | 6648(3) | 9544(2) | 203(1) | 28(1) | 1 |
| C25 | 4742(3) | 8877(2) | 787(1) | 29(1) | 1 |
| C26 | 4742(3) | 8877(2) | 787(1) | 29(1) | 1 |
| C27 | 7773(4) | 9610(2) | 630(1) | 25(1) | 1 |
| C28 | 7431(4) | 10628(2) | 868(1) | 29(1) | 1 |
| C29 | 9895(4) | 9489(2) | 569(1) | 36(1) | 1 |

TABLE C
Bond lengths [A] and angles [°].

| | | | |
|---|---|---|---|
| NI—CIO | 1.498(3) | C14—C15 | 1.518(3) |
| NI—CI5 | 1.522(3) | C16—C17 | 1.526(3) |
| NI—CII | 1.524(3) | C17—C18 | 1.527(4) |
| 01—C2 | 1.368(3) | C17—C19 | 1.527(4) |
| OI—CI | 1.432(3) | C20—C21 | 1.525(3) |
| 02—C7 | 1.369(3) | C20—S I | 1.784(2) |
| 02—C8 | 1.433(3) | SI—05 | 1.4442(19) |
| 03—C13 | 1.425(3) | SI—04 | 1.4607(17) |
| C2—C3 | 1.372(3) | SI—06 | 1.4676(18) |
| C2—C7 | 1.417(3) | 07—C22 | 1.208(3) |
| C3—C4 | 1.407(3) | C21—C22 | 1.537(4) |
| C4—C5 | 1.384(3) | C21—C26 | 1.559(3) |
| C4—C9 | 1.506(3) | C21—C27 | 1.565(3) |
| C5—C6 | 1.411(3) | C22—C23 | 1.517(4) |
| C5—C15 | 1.516(3) | C23—C24 | 1.535(4) |
| C6—C7 | 1.372(3) | C24—C25 | 1.548(4) |
| C9—CI0 | 1.504(3) | C24—C27 | 1.554(4) |
| CII—CI2 | 1.521(3) | C25—C26 | 1.557(4) |
| C12—C16 | 1.540(3) | C27—C28 | 1.529(3) |
| C12—C13 | 1.544(3) | C27—C29 | 1.542(4) |
| C13—C14 | 1.524(3) | | |
| CI0—NI—CI5 | 113.33(19) | CI2—CII—NI | 113.43(19) |
| CI0—NI—CII | 109.46(18) | CII—CI2—CI6 | 110.5(2) |
| CI5—NI—CII | 111.96(19) | CII—CI2—CI3 | 111.7(2) |
| C2—01—CI | 116.6(2) | CI6—CI2—CI3 | 109.84(19) |
| C7—02—C8 | 116.27(19) | 03—CI3—CI4 | 106.0(2) |
| 01—C2—C3 | 125.5(2) | 03—CI3—CI2 | 111.1(2) |
| 01—C2—C7 | 115.0(2) | CI4—CI3—CI2 | 111.0(2) |
| C3—C2—C7 | 119.5(2) | CI5—CI4—CI3 | 110.1(2) |
| C2—C3—C4 | 121.5(2) | C5—CI5—CI4 | 114.3(2) |
| C5—C4—C3 | 119.2(2) | C5—CI5—NI | 112.0(2) |
| C5—C4—C9 | 120.3(2) | CI4—CI5—NI | 108.7(2) |
| C3—C4—C9 | 120.5(2) | CI7—CI6—CI2 | 118.4(2) |
| C4—C5—C6 | 119.4(2) | CI6—CI7—CI8 | 112.2(2) |
| C4—C5—CI5 | 124.1(2) | CI6—CI7—CI9 | 108.7(2) |
| C6—C5—CI5 | 116.6(2) | CI8—CI7—CI9 | 110.8(3) |
| C7—C6—C5 | 121.3(2) | C21—C20—S1 | 122.51(18) |
| 02—C7—C6 | 125.4(2) | 05—SI—04 | 112.93(11) |
| 02—C7—C2 | 115.4(2) | 05—SI—06 | 112.47(12) |
| C6—C7—C2 | 119.2(2) | 04—SI—06 | 111.93(11) |
| CI0—C9—C4 | 111.7(2) | 05—SI—C20 | 108.81(13) |
| NI—CI0—C9 | 111.0(2) | 04—SI—C20 | 102.60(11) |
| 06—SI—C20 | 107.44(12) | C23—C24—C25 | 106.4(2) |
| C20—C21—C22 | 109.0(2) | C23—C24—C27 | 103.3(2) |
| C20—C21—C26 | 117.3(2) | C25—C24—C27 | 102.3(2) |
| C22—C21—C26 | 102.1(2) | C24—C25—C26 | 102.9(2) |
| C20—C21—C27 | 123.4(2) | C25—C26—C21 | 104.2(2) |
| C22—C21—C27 | 100.21(19) | C28—C27—C29 | 107.8(2) |
| C26—C21—C27 | 101.7(2) | C28—C27—C24 | 112.0(2) |
| 07—C22—C23 | 126.4(2) | C29—C27—C24 | 113.7(2) |
| 07—C22—C21 | 125.9(2) | C28—C27—C21 | 116.5(2) |
| C23—C22—C21 | 107.7(2) | C29—C27—C21 | 112.3(2) |
| C22—C23—C24 | 101.3(2) | C24—C27—C21 | 94.27(19) |

TABLE D
Anisotropic displacement parameters [A² × 10³].
The anisotropic displacement factor exponent takes the form:
−2π²[h²a*²U¹¹ + . . . + 2 h k a* b* U^{I2}].

| Atom | U^{II} | U²² | U³³ | U²³ | U^{I3} | U¹² |
|---|---|---|---|---|---|---|
| NI | 26(1) | 24(1) | 23(1) | 2(1) | −1(1) | −3(1) |
| 01 | 37(1) | 30(1) | 24(1) | 3(1) | −7(1) | −4(1) |
| 02 | 41(1) | 31(1) | 25(1) | 5(1) | −2(1) | −10(1) |
| 03 | 26(1) | 49(1) | 32(1) | 7(1) | −3(1) | −9(1) |
| CI | 41(2) | 36(2) | 32(2) | 3(1) | −9(1) | −8(2) |
| C2 | 30(2) | 24(2) | 22(1) | 1(1) | −1(1) | 2(1) |
| C3 | 25(1) | 26(1) | 24(1) | −3(1) | −2(1) | 2(1) |
| C4 | 26(2) | 22(1) | 23(1) | −1(1) | 2(1) | −1(1) |
| C5 | 24(1) | 22(1) | 23(1) | −2(1) | 1(1) | 0(1) |
| C6 | 26(1) | 22(1) | 24(1) | −3(1) | 2(1) | −5(1) |
| C7 | 30(2) | 22(1) | 22(1) | 2(1) | 4(1) | −4(1) |
| C8 | 45(2) | 34(2) | 36(2) | 5(1) | −2(1) | −20(2) |
| C9 | 23(1) | 32(1) | 29(2) | 3(1) | −1(1) | −4(1) |
| CIO | 26(1) | 29(1) | 25(1) | 2(1) | 0(1) | −5(1) |
| C11 | 31(1) | 25(1) | 20(1) | 2(1) | 0(1) | −2(1) |
| C12 | 26(1) | 26(1) | 23(1) | −1(1) | 1(1) | −1(1) |
| CI3 | 26(1) | 28(1) | 23(1) | −1(1) | −1(1) | −2(1) |
| CI4 | 30(2) | 22(2) | 24(1) | −1(1) | 1(1) | −1(1) |
| CI5 | 22(1) | 22(1) | 28(1) | 2(1) | 0(1) | −4(1) |
| C16 | 31(1) | 28(1) | 24(1) | −1(1) | −3(1) | 3(1) |
| CI7 | 46(2) | 31(2) | 25(1) | 1(1) | −7(1) | 0(2) |
| CI8 | 106(3) | 46(2) | 41(2) | 6(2) | −1(2) | 31(2) |
| C19 | 51(2) | 41(2) | 31(2) | 9(2) | −7(1) | −4(2) |
| C20 | 30(2) | 34(2) | 29(1) | 2(1) | 3(1) | 9(2) |
| S1 | 27(1) | 30(1) | 24(1) | 4(1) | −2(1) | −5(1) |
| O4 | 31(1) | 36(1) | 23(1) | 9(1) | −1(1) | 0(1) |
| O5 | 53(1) | 58(1) | 37(1) | 13(1) | −11(1) | −35(1) |
| O6 | 34(1) | 35(1) | 28(1) | −3(1) | −2(1) | 10(1) |
| O7 | 81(2) | 25(1) | 40(1) | −1(1) | 12(1) | 6(1) |
| C21 | 26(1) | 25(2) | 24(1) | −1(1) | 3(1) | 2(1) |
| C22 | 35(2) | 25(2) | 31(2) | 0(1) | 1(1) | −1(1) |
| C23 | 40(2) | 30(2) | 25(1) | −2(1) | 1(1) | −2(1) |
| C24 | 28(1) | 29(2) | 26(2) | 2(1) | 2(1) | 2(1) |
| C25 | 30(2) | 34(2) | 29(2) | −1(1) | −2(1) | 0(1) |
| C26 | 26(1) | 34(2) | 28(2) | 0(1) | 1(1) | −5(1) |
| C27 | 23(1) | 26(1) | 26(1) | 0(1) | 2(1) | 0(1) |
| C28 | 31(1) | 26(1) | 30(1) | 0(1) | −2(1) | −6(1) |
| C29 | 29(2) | 41(2) | 40(2) | 0(2) | 2(1) | −3(1) |

TABLE E
Hydrogen coordinates [×10⁴] and isotropic
displacement parameters [Å² × 10³].

| Atom | x | y | z | U_{eq} | S.o.f |
|---|---|---|---|---|---|
| H98 | 5190(40) | 10528(15) | 2062(10) | 70(8) | 1 |
| H99 | 10030(50) | 12950(30) | 2575(12) | 70(8) | 1 |
| H1A | 1107 | 11933 | 156 | 54 | 1 |
| H1B | 529 | 12973 | −89 | 54 | 1 |
| H1C | −154 | 12777 | 395 | 54 | 1 |
| H3 | 1220 | 11793 | 904 | 30 | 1 |
| H6 | 6760 | 13337 | 1353 | 29 | 1 |
| H8A | 6872 | 14966 | 1009 | 58 | 1 |
| H8B | 7600 | 15065 | 523 | 58 | 1 |
| H8C | 8193 | 14091 | 810 | 58 | 1 |
| H9A | 814 | 11106 | 1651 | 33 | 1 |
| H9B | 2505 | 10324 | 1567 | 33 | 1 |
| H10A | 2250 | 11767 | 2259 | 32 | 1 |
| H10B | 2235 | 10534 | 2304 | 32 | 1 |
| H11A | 4431 | 11494 | 2822 | 30 | 1 |
| H11B | 5322 | 10372 | 2759 | 30 | 1 |
| H12 | 8230 | 11108 | 2589 | 30 | 1 |
| H13 | 7334 | 13145 | 2840 | 30 | 1 |
| H14A | 4783 | 13050 | 2397 | 30 | 1 |
| H14B | 6354 | 13538 | 2090 | 30 | 1 |
| H15 | 7056 | 11776 | 1864 | 29 | 1 |
| H16A | 8973 | 11796 | 3278 | 33 | 1 |
| H16B | 6813 | 11911 | 3386 | 33 | 1 |
| I H17 | 6493 | 10098 | 3412 | 41 | 1 |
| H18A | 8906 | 9588 | 2944 | 97 | 1 |
| H18B | 9176 | 9031 | 3400 | 97 | 1 |
| H18C | 10440 | 10005 | 3276 | 97 | 1 |
| H19A | 9329 | 10894 | 3971 | 62 | 1 |
| H19B | 8110 | 9887 | 4073 | 62 | 1 |
| H19C | 7135 | 10999 | 4054 | 62 | 1 |
| H20A | 8824 | 7924 | 1207 | 37 | 1 |
| H20B | 6787 | 7484 | 1286 | 37 | 1 |
| H23A | 6070 | 8190 | −151 | 38 | 1 |
| H23B | 8277 | 8423 | −116 | 38 | 1 |
| H24 | 6928 | 10107 | −8 | 33 | 1 |
| H25A | 3773 | 9195 | 153 | 37 | 1 |
| H25B | 4152 | 10235 | 426 | 37 | 1 |
| H26A | 3994 | 8237 | 764 | 35 | 1 |
| H26B | 4300 | 9279 | 1039 | 35 | 1 |
| H28A | 8160 | 10638 | 1135 | 44 | 1 |
| I H28B | 6103 | 10692 | 936 | 44 | 1 |
| H28C | 7811 | 11207 | 684 | 44 | 1 |
| H29A | 10358 | 10042 | 381 | 54 | 1 |
| H29B | 10159 | 8817 | 436 | 54 | 1 |
| H29C | 10517 | 9531 | 849 | 54 | 1 |

TABLE 6
Hydrogen bonds [Å and °].

| D-H...A | d(D-H) | d(H...A) | d(D...A) | ∠(DHA) |
|---|---|---|---|---|
| N1-H98...O6 | 0.885(10) | 1.895(12) | 2.773(3) | 171(3) |
| N1-H98...S1 | 0.885(10) | 2.914(14) | 3.771(2) | 163(3) |
| O3-H99...O4ⁱ | 0.84(4) | 1.94(4) | 2.766(3) | 165(3) |
| O3-H99...S1ⁱ | 0.84(4) | 2.98(4) | 3.811(2) | 169(3) |

Symmetry transformations used to generate equivalent atoms: (i) −x + 2, y + ½, −z + ½

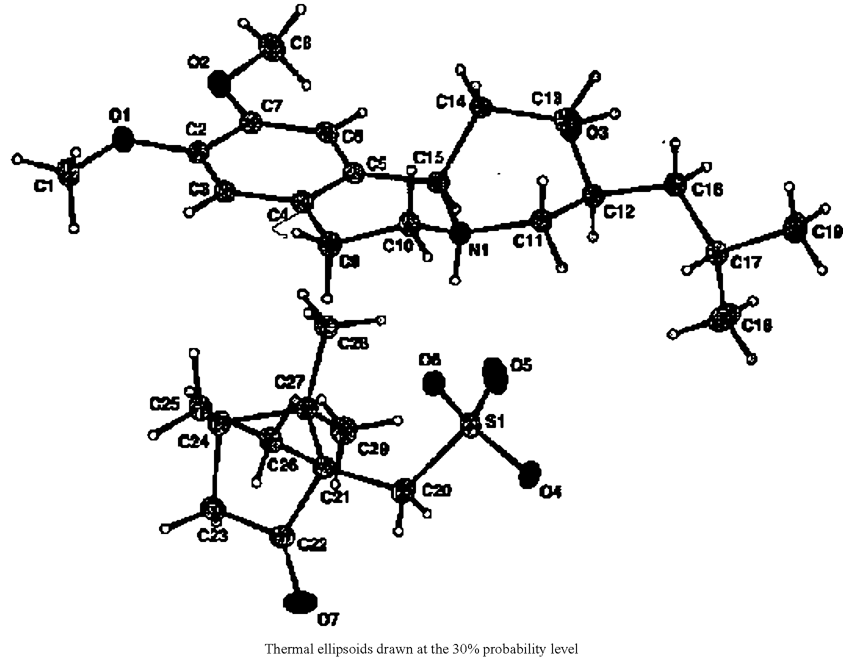

On the basis of the data set out above, Isomer B is believed to have the 2S, 3S, 11bR configuration, which corresponds to Formula (Ia):

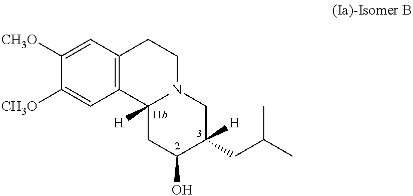

### Example 4

### A Study of the Effect of the Compound of Formula (Ia) in an Experimental Autoimmune Encephalomyelitis (EAE) Model of Multiple Sclerosis

Groups of 10 Biozzi mice aged between 5 and 8 weeks of age were challenged in the flank with mouse spinal cord homogenate (SCH) in complete Freund's adjuvant (CFA) in order to induce EAE. Treatments were given according to the schedule below.

Treatment (n=8 per group)
Group A—vehicle control
Group B—RU350 30 mg/kg days −1 to 10 once daily by oral gavage
Group C—RU350 10 mg/kg days −1 to 10 twice daily by oral gavage
Group D—Positive control drug

A 20 mg/ml solution of SCH in PBS was prepared and mixed in an equal volume with CFA. The mixture was sonicated in a Branson 1200 sonicator for 15 minutes and then homogenised for three minutes. An amount of 1 mg in 100 μl was administered per mouse by subcutaneous injection at the base of tail. In addition, mice were given 200 ng pertussis toxin in 0.5 ml by intraperitoneal (ip) injection on day 0 and again 48 hours later.

Mice were treated according to the outline above from day −1 to day 10. The compound of formula (Ia) (i.e. RUS350) was prepared daily by dissolving 15 mg of the compound (stored at −20° C.) in sterile distilled water to give 6 mg/ml solution. A dose of 100 μl was given by oral gavage daily to the group B mice (equivalent to 30 mg/kg per 20 g mouse). For group C mice, 1 ml of the 6 mg/kg stock was further diluted 1:3 in sterile distilled water, and a dose of 100 μl was given orally twice daily (equivalent to 2×10 mg/kg per 20 g mouse). Distilled water served as the vehicle control for the mice in group A. Group D mice were given a dose of 5 mg/kg dexamethasone (100 μg in 100 μl per 20 g mouse) on days 3-7 and days 10-12.

Moribund animals were euthanised according to Home Office regulations and all remaining animals were euthanized at day 28 post-induction. At termination, brains and spinal cords were taken from all animals and fixed for histopathology examination.

Mice were weighed prior to EAE induction (Day 0) and then daily from day 5 until termination. Clinical disease was monitored from day 5 and scored according the following system:

| | |
|---|---|
| 0 | normal |
| 0.5 | weight loss without clinical symptoms |
| 1 | flaccid paralysis of tail |
| 1.5 | impairment of the righting reflex |
| 2 | single hind limb paralysis |
| 3 | bilateral hind limb paralysis |
| 4 | hind limb and forelimb paralysis |
| 5 | moribund |

The results are shown in FIGS. 1 and 2.

The results show that the untreated animals developed severe EAE associated with weight loss and that treatment with steroid markedly inhibited this effect. The test compound of formula (Ia) also had a profound effect on the disease. A moderate degree of protection was afforded by a single 30 mg/kg dose of the test compound given daily. However, the 2× daily dose of 10 mg/kg gave a very high level of protection, analogous to that afforded by the steroid.

The data therefore show that the compound of formula (Ia) is potentially of use as an alternative to steroids in treating MS in humans.

### Example 5

### Pharmaceutical Compositions

### (i) Tablet Formulation—I

A tablet composition containing a dihydrotetrabenazine of the invention is prepared by mixing 50 mg of the dihydrotetrabenazine with 197 mg of lactose (BP) as diluent, and 3 mg magnesium stearate as a lubricant and compressing to form a tablet in known manner.

### (ii) Tablet Formulation—II

A tablet composition containing a dihydrotetrabenazine of the invention is prepared by mixing the compound (25 mg) with iron oxide, lactose, magnesium stearate, starch maize white and talc, and compressing to form a tablet in known manner.

(iii) Capsule Formulation

A capsule formulation is prepared by mixing 100 mg of a dihydrotetrabenazine of the invention with 100 mg lactose and filling the resulting mixture into standard opaque hard gelatin capsules.

### EQUIVALENTS

It will readily be apparent that numerous modifications and alterations may be made to the specific embodiments of the invention described above without departing from the principles underlying the invention. All such modifications and alterations are intended to be embraced by this application.

1-14. (canceled)

15. A method of treating multiple sclerosis or an autoimmune myelitis, which method comprises administering to a patient in need thereof, an effective treatment amount of a 3, 11b-cis-dihydrotetrabenazine compound of formula (Ia): 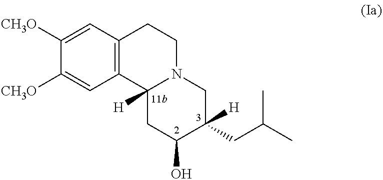
or a pharmaceutically acceptable salt thereof.

16. The method of claim 15, wherein the 3,11b-cis-dihydrotetrabenazine is in the form of an acid addition salt.

17. The method of claim 16, wherein the acid addition salt is a methane sulphonate salt.

18. The method of claim 15, wherein the 3,11b-cis-dihydrotetrabenazine of the formula (Ia) or pharmaceutically acceptable salt thereof has an isomeric purity of greater than 90%.

19. The method of claim 15, wherein the 3,11b-cis-dihydrotetrabenazine of the formula (Ia) or pharmaceutically acceptable salt thereof has an isomeric purity of greater than 98%.

20. The method of claim 15, wherein the method is for treating multiple sclerosis.

21. The method of claim 15, wherein the method is for treating an autoimmune myelitis.

22. The method of claim 15, wherein the treatment consists of or comprises any one or more of:
halting the progression of the disease;
slowing the progression of the disease;
modifying the progression of the disease;
providing symptomatic relief, e.g. by eliminating or reducing the severity of one or more symptoms;
extending periods of remission;
preventing relapses;
reducing the severity of relapses; and
preventing or slowing the progression from an initial period of relapsing-remitting MS to secondary progressive MS.

23. The method of claim 15, wherein the treatment consists of or comprises the elimination, amelioration or reduction in severity of any one or more symptoms, in any combination, selected from:
weakness and/or numbness in one or more extremities;
tingling of the extremities;
tight band-like sensations around the trunk or limbs;
tremor of one or more extremities;
dragging or poor control of one or both legs;
spastic or ataxic paraparesis;
paralysis of one or more extremities;
hyperactive tendon reflexes;
disappearance of abdominal reflexes;
Lhermitte's sign;
retrobulbar or optic neuritis;
unsteadiness in walking;
problems with balance,
increased muscle fatigue;
brain stem symptoms (diplopia, vertigo, vomiting);
disorders of micturition;
hemiplegia;
trigeminal neuralgia;
other pain syndromes;
nystagmus and ataxia;
cerebellar-type ataxia;
Charcot's triad; diplopia;
bilateral internuclear ophthalmoplegia;
myokymia or paralysis of facial muscles;
deafness;
tinnitus;
unformed auditory hallucinations (because of involvement of cochlear connections);
transient facial anesthesia or of trigeminal neuralgia;
urinary and/or faecal incontinence
bladder dysfunction euphoria;
depression;
fatigue;
dementia;
dull, aching pain in the low back;
sharp, burning, poorly localized pains in a limb;
abrupt attacks of neurologic deficit;
dysarthria and ataxia;
paroxysmal pain and dysesthesia in a limb;
flashing lights;
paroxysmal itching;
tonic seizures;
changes in sensation;
visual problems;
muscle weakness;
difficulties with coordination and speech;
cognitive impairment;
overheating; and
impaired mobility and disability.

24. The method of claim 15, wherein the treatment is a prophylactic treatment.

25. The method of claim 15, wherein the prophylactic treatment comprises administration of the compound during periods of remission in order to prevent or reduce the likelihood or severity of relapses.

26. A compound: 3, 11b-cis-dihydrotetrabenazine of formula (Ia): 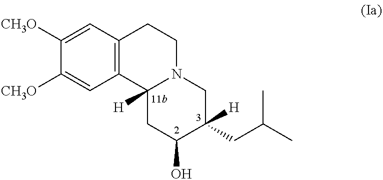
or a pharmaceutically acceptable salt thereof.

27. A composition comprising an effective treatment amount of a 3, 11b-cis-dihydrotetrabenazine compound of formula (Ia): 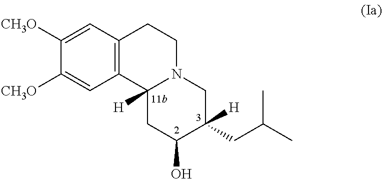
or a pharmaceutically acceptable salt thereof.

28. The composition of claim 27, wherein the 3,11b-cis-dihydrotetrabenazine is in the form of an acid addition salt.

29. The composition of claim 28, wherein the acid addition salt is a methane sulphonate salt.

30. The composition of claim 27, wherein the 3,11b-cis-dihydrotetrabenazine of the formula (Ia) or pharmaceutically acceptable salt thereof has an isomeric purity of greater than 90%.

31. The composition of claim 27, wherein the 3,11b-cis-dihydrotetrabenazine of the formula (Ia) or pharmaceutically acceptable salt thereof has an isomeric purity of greater than 98%.

32. The composition of claim 27, further comprising an inert diluent or carrier.

33. The composition of claim 27, wherein the effective treatment amount is effective for treating multiple sclerosis or an autoimmune myelitis.

34. The composition of claim 27, wherein the effective treatment amount is effective for any one or more of:
halting the progression of the disease;
slowing the progression of the disease;
modifying the progression of the disease;
providing symptomatic relief, e.g. by eliminating or reducing the severity of one or more symptoms;
extending periods of remission;
preventing relapses;
reducing the severity of relapses; and
preventing or slowing the progression from an initial period of relapsing-remitting MS to secondary progressive MS.

35. The composition of claim 27, wherein the effective treatment amount is effective for elimination, amelioration or reduction in severity of any one or more symptoms, in any combination, selected from:
weakness and/or numbness in one or more extremities;
tingling of the extremities;
tight band-like sensations around the trunk or limbs;
tremor of one or more extremities;
dragging or poor control of one or both legs;
spastic or ataxic paraparesis;
paralysis of one or more extremities;
hyperactive tendon reflexes;
disappearance of abdominal reflexes;
Lhermitte's sign;
retrobulbar or optic neuritis;
unsteadiness in walking;
problems with balance,
increased muscle fatigue;
brain stem symptoms (diplopia, vertigo, vomiting);
disorders of micturition;
hemiplegia;
trigeminal neuralgia;
other pain syndromes;
nystagmus and ataxia;
cerebellar-type ataxia;
Charcot's triad; diplopia;
bilateral internuclear ophthalmoplegia;
myokymia or paralysis of facial muscles;
deafness;
tinnitus;
unformed auditory hallucinations (because of involvement of cochlear connections);
transient facial anesthesia or of trigeminal neuralgia;
urinary and/or faecal incontinence
bladder dysfunction euphoria;
depression;
fatigue;
dementia;
dull, aching pain in the low back;
sharp, burning, poorly localized pains in a limb;
abrupt attacks of neurologic deficit;
dysarthria and ataxia;
paroxysmal pain and dysesthesia in a limb;
flashing lights;
paroxysmal itching;
tonic seizures;
changes in sensation;
visual problems;
muscle weakness;
difficulties with coordination and speech;
cognitive impairment;
overheating; and
impaired mobility and disability.
